(19) United States
(12) Patent Application Publication
(10) Pub. No.: US 2005/0053568 A1
(43) Pub. Date: Mar. 10, 2005

(54) Composition containing a wax, and used thereof

(75) Inventors: Odile Aubrun-Sonneville, Antony (FR); Florence L'Alloret, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/813,098

(22) Filed: Mar. 31, 2004

Related U.S. Application Data
(60) Provisional application No. 60/470,419, filed on May 15, 2003.

(30) Foreign Application Priority Data
Apr. 11, 2003 (FR) 0304577

(51) Int. Cl. A61K 7/06; A61K 7/11

(52) U.S. Cl. 424/70.16

(57) ABSTRACT

The invention relates to a composition for topical application in the form of an O/W emulsion containing at least one wax and at least one particular amphiphilic polymer, and to the use of the composition, in particular for caring for, protecting and/or making up body or facial skin, the eyelashes and/or the lips, and/or for haircare.

### REFERENCE TO PRIOR APPLICATIONS

This application claims priority to U.S. provisional application 60/470,419 filed May 15, 2003 and to French patent application 0304577 filed Apr. 11, 2003, both incorporated herein by reference.

### FIELD OF THE INVENTION

The invention relates to a composition in the form of an O/W emulsion containing at least one wax and at least one amphiphilic polymer, and to the use of the said composition, in particular for caring for, protecting and/or making up body or facial skin, the eyelashes and/or the lips, and/or for haircare. Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

In the cosmetics field, the addition of waxes to skincare compositions produces products that are particularly suitable for treating dry skin and elderly skin. Waxes contribute especially towards nutrifying the skin and smoothing out wrinkles and fine lines. They may be introduced either into W/O emulsions or into O/W emulsions. In general, in the cosmetics field, O/W emulsions are preferred to W/O emulsions on account of their cosmetic qualities, which are considered as being superior (lightness and freshness), and their greater formulation flexibility. However, the presence of waxes in standard O/W formulations results in impairment in their stability, unlike W/O formulations. Moreover, the cosmetic properties of O/W formulations containing waxes are still considered to be insufficient with regard to certain criteria: excessively high consistency, difficulty of spreading and presence of a greasy film.

There is thus still a need to produce stable O/W emulsions containing waxes, which have improved cosmetic properties.

### DETAILED DESCRIPTION OF THE PREFERRED EMBODIMENTS

The inventors have discovered, unexpectedly, that the use of particular non-crosslinked polymers, derived from 2-acrylamido-2-methylpropanesulphonic acid (AMPS), solves the problem underlying the invention and provides a stable composition that has good cosmetic properties and that may optionally but advantageously be free of emulsifying surfactant conventionally used in O/W emulsions, and that is thus well tolerated by any type of skin.

EP-A-1 069 142 discloses O/W emulsions containing AMPS-based polymers comprising a hydrophobic chain. However, the O/W emulsions described in this document do not contain waxes whose presence is a destabilizing factor on the emulsion. In addition, the use of waxes is only described in the document in Example 45, which not only is a W/O emulsion rather than an O/W emulsion but also contains an emulsifier (polyglyceryl-2 sesquistearate), which can become positioned at the interface between the aqueous and oily phases, the non-crosslinked AMPS polymer being present as thickener.

Moreover, documents WO-A-02/43689, WO-A-02/44231, WO-A-02/44271, WO-A-02/44270, WO-A-02/43686, WO-A-02/44267 and WO-A-02/44230 describe cosmetic, dermatological or pharmaceutical compositions for topical application, comprising an AMPS-based amphiphilic copolymer. However, none of the documents describes O/W emulsions containing waxes.

The inventors have found, surprisingly, that the use of particular non-crosslinked polymers described below, characterized by given proportions of monomers, makes it possible to achieve the aim of the invention, i.e. to obtain O/W emulsions containing waxes, which are stable even in the absence of standard surfactants for O/W emulsions.

Moreover, some of the polymers used according to the invention produce compositions that have particularly pleasant textures and thus very good cosmetic properties. However, these more particular polymers do not by themselves make it possible to obtain stable emulsions, and the inventors have found, surprisingly, that the combination of these polymers with one or more waxes solves this problem of instability of the emulsion.

Thus, not only can the selected polymers produce emulsions containing waxes, which are stable even in the absence of standard surfactants, but also the waxes can stabilize emulsions containing some of these polymers, which have the advantage of giving emulsions with a very cosmetic texture, but which have the drawback of not being sufficient by themselves to obtain a stable emulsion. The combination of at least one wax and of at least one polymer used according to the invention is thus particularly advantageous for obtaining emulsions that are both stable and endowed with good cosmetic properties.

One subject of the invention is thus a composition for topical application in the form of an O/W emulsion comprising an oily phase dispersed in an aqueous phase, wherein it contains at least one wax and at least one non-crosslinked amphiphilic polymer comprising from:
(a) 80 mol % to 99 mol % of 2-acrylamido-2-methylpropane-sulphonic acid (AMPS) units of formula (I) below: 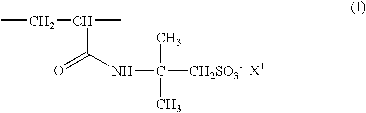
in which X⁺ is a proton, an alkali metal cation, an alkaline-earth metal cation, an ammonium ion or an organic cation; and
(b) 1 mol % to 20 mol % of units of formula (II) below: 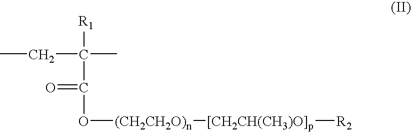
in which n and p, independently of each other, denote an integer ranging from 0 to 24, preferably from 1 to 24 and more preferably from 3 to 20, with the proviso that n+p is less than 25, preferably less than 20 and better still less than 15; R₁ denotes a hydrogen atom or a linear or branched alkyl radical containing from 1 to 6 carbon atoms (C₁-C₆) (preferably methyl), and R₂ denotes a linear or branched alkyl radical containing from 6 to 30 carbon atoms (C₆-C₃₀), preferably from 6 to 22 (C₆-C₂₂) and more preferably from 12 to 18 carbon atoms (C₁₂-C₁₈).

Since the composition according to the invention is intended for topical application to the skin or the integuments, it preferably contains a physiologically acceptable medium. The term “physiologically acceptable medium” means a medium that is compatible with the skin, the lips, the nails, the scalp and/or the hair.

The composition obtained according to the invention shows good stability over time, even at a temperature above room temperature (for example 45° C.). The term “stable emulsions” means emulsions which, after storage for 24 hours at all temperatures between 4° C. and 50° C., show no macroscopic change in colour, odour or viscosity and no variation in pH.

The composition of the invention is preferably in the form of a more or less fluid cream or a milk, i.e. a supple product, as opposed to a solid product such as a stick. Thus, this preferred composition form may have a viscosity at room temperature (25° C.) ranging from about 1 to 250 poises (0.1 to 25 Pa.s) and preferably from about 1 to 150 poises (1 to 15 Pa.s), this viscosity being measured with a Rheomat 180 machine at 25° C.

Non-Crosslinked Polymers

The polymers used in the composition of the invention are non-crosslinked copolymers based on at least one hydrophilic AMPS monomer of formula (I) and on at least one hydrophobic monomer of formula (II). These polymers are water-soluble or water-dispersible, in neutralized or partially neutralized form. They are preferably totally neutralized. Since the polymers are water-soluble, they are introduced into the aqueous phase of the emulsion.

The expression “water-soluble or water-dispersible polymers” means polymers which, when introduced into water to a concentration equal to 1% by weight, give a macroscopically homogeneous solution whose light transmittance, at a wavelength equal to 500 nm, through a sample 1 cm thick, is at least 10%.

The term “non-crosslinked polymer” means a polymer that has not reacted with crosslinking agents and that therefore does not comprise crosslinking groups. This is a linear polymer.

The polymers of the invention preferably have a weight-average molecular weight ranging from 10,000 to 10,000,000, more preferably from 100,000 to 8,000,000 and even more preferably from 100,000 to 7,000,000.

More particularly, 2-acrylamido-2-methyl-propanesulphonic acid (AMPS) of formula (I) is used, in its partially or totally neutralized forms.

Preferably, the polymers in accordance with the invention are partially or totally neutralized with a mineral base (sodium hydroxide, potassium hydroxide or aqueous ammonia) or an organic base such as mono-ethanolamine, diethanolamine or triethanolamine, an aminomethylpropanediol, N-methylglucamine, basic amino acids, for instance arginine and lysine, and mixtures of these compounds.

The polymers of the invention may be chosen especially from the non-crosslinked amphiphilic polymers corresponding to the definition given above, described in general in document EP-A-1 069 142.

As indicated above, some of these polymers are more particularly advantageous for obtaining a composition that has superior cosmetic properties. These are more particularly polymers comprising from: (a) 85 mol % to 99 mol % and preferably from 90 mol % to 99 mol % of 2-acrylamido-2-methylpropanesulphonic acid (AMPS) units of formula (I) as indicated above, and (b) 1 mol % to 15 mol % and preferably from 1 mol % to 10 mol % of units of formula (III) below: 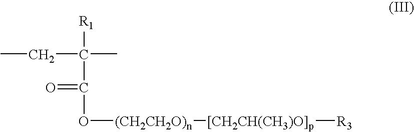
in which n and p, independently of each other, denote an integer ranging from 7 to 24 and preferably from 8 to 24, with the proviso that n+p is less than 25, better still less than 20 and even better still less than 15; R₁ denotes, as in formula (II), a hydrogen atom or a linear or branched alkyl radical containing from 1 to 6 carbon atoms (C₁-C₆) (preferably methyl) and R₃ denotes a linear or branched alkyl radical containing from 6 to 15 carbon atoms (C₆-C₁₅) and preferably from 8 to 14 carbon atoms (C₈-C₁₄).

The amphiphilic polymers used according to the invention may be obtained according to a standard free-radical polymerization processes in the presence of one or more initiators such as, for example, azobisisobutyronitrile (AIBN), azobisdimethyl-valeronitrile, ABAH (2,2-azobis[2-amidinopropane] hydrochloride), organic peroxides such as dilauryl peroxide, benzoyl peroxide, tert-butyl hydroperoxide, etc., mineral peroxide compounds such as potassium persulphate or ammonium persulphate, or H₂O₂ optionally in the presence of reducing agents.

The polymers may be obtained especially by free-radical polymerization in tert-butanol medium in which they precipitate. Using precipitation polymerization in tert-butanol, it is possible to obtain a size distribution of the polymer particles that is particularly favourable for its uses.

The polymerization reaction may be performed at a temperature of between 0 and 150° C., preferably between 10 and 100° C., either at atmospheric pressure or under reduced pressure. It may also be performed under inert atmosphere, and preferably under nitrogen.

According to this process, polymers used according to the invention were obtained especially from 2-acrylamido-2-methylpropanesulphonic acid (AMPS) or a sodium or ammonium salt thereof with a (meth)acrylic acid ester, preferably a methacrylic acid ester, and
a C₁₀-C₁₈ alcohol oxyethylenated with 8 mol of ethylene oxide (Genapol® C-080 from the company Hoechst/Clariant),
a C₁₁ oxo alcohol oxyethylenated with 8 mol of ethylene oxide (Genapol® UD-080 from the company Hoechst/Clariant),
a C₁₁ oxo alcohol oxyethylenated with 7 mol of ethylene oxide (Genapol® UD-070 from the company Hoechst/Clariant),
a C₁₂-C₁₄ alcohol oxyethylenated with 7 mol of ethylene oxide (Genapol® LA-070 from the company Hoechst/Clariant),
a C₁₂-C₁₄ alcohol oxyethylenated with 9 mol of ethylene oxide (Genapol® LA-090 from the company Hoechst/Clariant),
a C₁₂-C₁₄ alcohol oxyethylenated with 11 mol of ethylene oxide (Genapol® LA-110 from the company Hoechst/Clariant),
a C₁₆-C₁₈ alcohol oxyethylenated with 8 mol of ethylene oxide (Genapol® T-080 from the company Hoechst/Clariant),
a C₁₆-C₁₈ alcohol oxyethylenated with 15 mol of ethylene oxide (Genapol® T-150 from the company Hoechst/Clariant),
a C₁₆-C₁₈ alcohol oxyethylenated with 11 mol of ethylene oxide (Genapol® T-110 from the company Hoechst/Clariant),
a C₁₆-C₁₈ alcohol oxyethylenated with 20 mol of ethylene oxide (Genapol® T-200 from the company Hoechst/Clariant).

Preferred amphiphilic polymers used according to the present invention include the copolymer obtained from 91.5 mol % of AMPS and 8.5 mol % of a C₁₂-C₁₄ alkyl methacrylate comprising 7 oxyethylene groups (Genapol LA-070) (polymer I); the copolymer obtained from 92.65 mol % of AMPS and 7.35 mol % of a C₁₆-C₁₈ alkyl methacrylate comprising 8 oxyethylene groups (Genapol T-080) (polymer II); and a blend of these polymers. These polymers are all suitable for giving a composition that is stable in the presence of wax. These polymers can also produce a composition that has particularly pleasant cosmetic properties. Thus, the creams obtained are very pleasant to apply and leave a film that is neither greasy nor sticky on the skin. Furthermore, they leave the skin surface velvety and have good moisturizing properties.

The amount of amphiphilic polymer(s) in the composition of the invention is not particularly limited and generally depends on the polymer used. It can range, for example, in terms of active material, from 0.05% to 20% by weight, preferably from 0.1% to 15% by weight, better still from 0.2% to 10% by weight and even better still from 0.25% to 5% by weight relative to the total weight of the composition.

According to one particular embodiment of the invention, the composition according to the invention may be substantially free of emulsifying surfactant usually used in O/W emulsions. The expression “substantially free of surfactant” means a composition containing less than 0.5% by weight and preferably less than 0.3% by weight of emulsifying surfactant relative to the total weight of the composition, or even being totally free of any emulsifier.

Oily Phase

The composition of the invention comprises at least one wax, which is generally present in the oily phase. The amount of wax(es) in the composition of the invention is not particularly limited and can range, for example, from 0.05% to 10% by weight, better still from 0.1% to 15% by weight and even better still from 0.5% to 10% by weight relative to the total weight of the composition.

The term “waxes” means fatty substances that are solid at room temperature (25° C.) and that have a melting point of greater than 30° C. (measured by DSC), and better still greater than 400° C.

For the purposes of the patent application, the waxes include those generally used in cosmetics and dermatology; they may be hydrocarbon-based waxes, silicone waxes and/or fluoro waxes, optionally comprising ester or hydroxyl functions. It is possible, for example, to use mineral waxes; waxes of animal origin; waxes of plant origin; hydrogenated oils that are solid at 25° C. , fatty esters and glycerides that are solid at 25° C., synthetic waxes and silicone waxes, and mixtures thereof.

As examples of waxes that may be used in the composition of the invention, particular mention may be made of microcrystalline waxes; paraffin waxes; lignite waxes; ceresin; ozokerite; montan wax; beeswax; lanolin and its derivatives; candelilla wax, ouricury wax, carnauba wax and Japan wax; cocoa butter; palm oil in paste form at 20° C.; cork fibre wax or sugarcane wax; hydrogenated oils that are solid at 25° C.; fatty esters and glycerides that are solid at 25° C.; polyethylene waxes and the waxes obtained by Fischer-Tropsch synthesis; silicone waxes, and mixtures thereof.

According to one preferred embodiment of the invention, at least one wax chosen from carnauba wax, beeswax, palm oil in paste form at 20° C., polyethylene waxes with a starting melting point of greater than 65° C. and microcrystalline waxes with a starting melting point of greater than 65° C., and mixtures thereof, is used.

Besides the waxes, the oily phase of the composition according to the invention may contain at least one fatty substance chosen from volatile or non-volatile oils that are liquid at room temperature (20-25° C.), gums and pasty fatty substances, of animal, plant, mineral or synthetic origin, and mixtures thereof. These fatty substances are physiologically acceptable.

According to one preferred embodiment of the invention, the oily phase contains at least one oil. The term “oil” means a fatty substance that is liquid at room temperature (25° C.).

As oils that may be used in the composition of the invention, examples that may be mentioned include:
hydrocarbon-based oils of animal origin, such as perhydrosqualene (or squalane);
hydrocarbon-based oils of plant origin, such as liquid triglycerides of fatty acids containing from 4 to 10 carbon atoms, for instance heptanoic or octanoic acid triglycerides, or alternatively, for example, sunflower oil, maize oil, soybean oil, marrow oil, grapeseed oil, sesame seed oil, hazelnut oil, apricot oil, macadamia oil, coriander oil, arara oil, castor oil, avocado oil, caprylic/capric acid triglycerides, for instance those sold by the company Stearineries Dubois or those sold under the names Miglyol 810, 812 and 818 by the company Dynamit Nobel, jojoba oil, shea butter oil and liquid fractions of shea butter;
synthetic esters and ethers, especially of fatty acids, for instance the oils of formulae R¹COOR² and R¹OR² in which R¹ represents a fatty acid residue containing from 8 to 29 carbon atoms and R² represents a branched or unbranched hydrocarbon-based chain containing from 3 to 30 carbon atoms, for instance purcellin oil, isononyl isononanoate, isopropyl myristate, 2-ethylhexyl palmitate (or octyl palmitate), 2-octyldodecyl stearate, 2-octyldodecyl erucate or isostearyl isostearate; hydroxylated esters, for instance isostearyl lactate, octyl hydroxystearate, octyldodecyl hydroxystearate, diisostearyl malate, triisocetyl citrate, and fatty alkyl heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tetraisostearate; lipophilic amino acid derivatives, such as isopropyl lauroyl sarcosinate (INCI name: Isopropyl lauroyl sarcosinate) sold under the name Eldew SL 205 by the company Ajinomoto;
linear or branched hydrocarbons of mineral or synthetic origin, such as mineral oils (mixture of hydrocarbon-based oils derived from petroleum; INCI name: Mineral oil), volatile or non-volatile liquid paraffins, and derivatives thereof, petroleum jelly, polydecenes, isohexadecane, isododecane, and hydrogenated isoparaffin such as Parleam® oil, sold by the company NOF Corporation (INCI name: Hydrogenated polyisobutene);
fatty alcohols containing from 8 to 26 carbon atoms, for instance cetyl alcohol, stearyl alcohol and the mixture thereof (cetearyl alcohol), octyldodecanol, 2-butyloctanol, 2-hexyldecanol, 2-undecylpentadecanol or oleyl alcohol;
partially hydrocarbon-based and/or silicone-based fluoro oils, for instance those described in document JP-A-2 295 912;
silicone oils, for instance volatile or non-volatile polymethylsiloxanes (PDMSs) containing a linear or cyclic silicone chain, which are liquid or pasty at room temperature, especially volatile silicone oils which are either linear or cyclic, for instance cyclopolydimethylsiloxanes (cyclomethicones) such as cyclopentasiloxane and cyclohexadimethylsiloxane; polydimethylsiloxanes comprising alkyl, alkoxy or phenyl groups, which are pendent or at the end of a silicone chain, these groups containing from 2 to 24 carbon atoms; phenylsilicones, for instance phenyltrimethicones, phenyldimethicones, phenyltrimethylsiloxydiphenylsiloxanes, diphenyl-dimethicones, diphenylmethyldiphenyltrisiloxanes, 2-phenylethyltrimethylsiloxysilicates and polymethyl-phenylsiloxanes;
mixtures thereof.

According to one preferred embodiment of the invention, the oily phase of the composition contains at least one oil chosen from linear or branched hydro-carbons of mineral or synthetic origin, such as parleam oil, from volatile silicone oils, especially cyclopoly-dimethylsiloxanes, from plant oils, such as the liquid fractions of shea butter, and from esters, such as octyl palmitate.

The other fatty substances that may be present in the oily phase are, for example, fatty acids containing from 8 to 30 carbon atoms, for instance stearic acid, lauric acid or palmitic acid; gums such as silicone gums (dimethiconol); silicone resins such as trifluoromethyl-C₁₋₄ alkyldimethicone and trifluoropropyldimethicone, and silicone elastomers, for instance the products sold under the name “KSG” by the company Shin-Etsu, under the name “Trefil” by the company Dow Corning or under the name “Gransil” by the company Grant Industries; pastes such as Petrolatum; and mixtures thereof.

According to one particular embodiment of the invention, the composition contains at least one silicone gum whose presence further improves the cosmetic qualities of the composition. One or more silicone gums with a molecular weight of less than 1,500,000 are preferably used, such as a polydimethyl-siloxane (INCI name: dimethicone), a polyphenylsiloxane or a polyhydroxysiloxane (INCI name: dimethiconol). When it is present, the amount of silicone gum can range, for example, from 0.01% to 10% by weight and preferably from 0.1% to 3% by weight relative to the weight of the final composition.

The oily phase comprising all the fatty substances and the lipophilic adjuvants that may be present is present in the composition according to the invention in an amount generally ranging from 10% to 75% and preferably from 15% to 70% by weight relative to the total weight of the composition.

Aqueous Phase

The aqueous phase of the composition of the invention is not particularly limited and generally may range from 90% to 25% by weight and preferably from 85% to 30% by weight relative to the total weight of the composition. It contains at least water. Besides water, it may contain one or more water-miscible or at least partially water-miscible compounds, for instance polyols; C₂ to C₈ lower monoalcohols, such as ethanol and isopropanol; and C₃ to C₄ ketones that are liquid at room temperature. The term “room temperature” should be understood as meaning a temperature of about 25° C. at normal atmospheric pressure (760 mmHg).

The term “polyol” should be understood as meaning any organic molecule comprising at least two free hydroxyl groups. Examples of polyols that may be mentioned include glycerol, glycols, for instance butylene glycol, propylene glycol, isoprene glycol, dipropylene glycol, hexylene glycol, pentylene glycol and polyethylene glycols, for instance PEG-8, sorbitol and sugars, for instance glucose.

The solvent(s) may be present in an amount ranging from 0.1% to 30% by weight relative to the total weight of the composition.

Adjuvants

The composition of the invention may also contain adjuvants that are common in cosmetics and/or dermatology, such as active agents, preserving agents, antioxidants, complexing agents, pH regulators (acidic or basic), fragrances, fillers, bactericides, odour absorbers, dyestuffs (pigments and dyes), surfactants, polymers or lipid vesicles. The amounts of these various adjuvants are those conventionally used in the field under consideration, for example from 0.01% to 20% of the total weight of the composition. Depending on their nature, these adjuvants may be introduced into the fatty phase, into the aqueous phase and/or into the lipid vesicles.

As polymers that may additionally be used in the composition according to the invention, other than the polymers used according to the invention as mentioned above, mention may be made especially of hydrophilic polymers such as modified or unmodified carboxyvinyl polymers, such as the products sold under the names Carbopol (INCI name: Carbomer) and Pemulen (INCI name: Acrylates/C10-30 alkyl acrylate crosspolymer) by the company Goodrich; polyacrylamides; polymers derived from 2-acrylamido-2-methylpropanesulphonic acid, such as the product sold by Clariant under the name Hostacerin AMPS (INCI name: ammonium polyacryldimethyltaurate); crosslinked anionic copolymers of acrylamide and of AMPS, in the form of an emulsion, such as those sold under the name Sepigel 305 (CTFA name: Polyacrylamide C13-14 Isoparaffin/-Laureth-7) and under the name Simulgel 600 (CTFA name: Acrylamide/Sodium acryloyldimethyltaurate copolymer/-Isohexadecane/Polysorbate 80) by the company SEPPIC; acrylate/acrylonitrile copolymers such as HYPAN SS201 sold by the company Kingston; synthetic neutral polymers such as poly-N-vinylpyrrolidone; polysaccharides, for instance guar gum, xanthan gum and cellulose derivatives; water-soluble or water-dispersible silicone derivatives, for instance acrylic silicones, polyether silicones and cationic silicones; mineral compounds such as modified or unmodified hectorites and smectites, such as the Bentone products sold by the company Rheox, the Laponite products sold by the company Southern Clay Products, the product Veegum HS sold by the company R. T. Vanderbilt; and mixtures thereof. The amount of these polymers may range, for example, from 0.05% to 3% by weight relative to the total weight of the composition.

As fillers that may be used in the composition of the invention, examples that may be mentioned include the pigments such as titanium oxide, zinc oxide or iron oxide and organic pigments; kaolin; silica; talc; boron nitride; organic spherical powders, fibres; and mixtures thereof. Examples of organic spherical powders that may be mentioned include polyamide powders and especially Nylon® powders such as Nylon-1 or Polyamide 12, sold under the name Orgasol by the company Atochem; polyethylene powders; Teflon®; microspheres based on acrylic copolymers, such as those made of ethylene glycol dimethacrylate/lauryl methacrylate copolymer sold by the company Dow Corning under the name Polytrap; expanded powders such as hollow microspheres and especially the microspheres sold under the name Expancel by the company Kemanord Plast or under the name Micropearl F 80 ED by the company Matsumoto; silicone resin microbeads such as those sold under the name Tospearl by the company Toshiba Silicone; polymethyl methacrylate microspheres, sold under the name Microsphere M-100 by the company Matsumoto or under the name Covabead LH85 by the company Wackherr; ethylene acrylate copolymer powders, such as those sold under the name Flobeads by the company Sumitomo Seika Chemicals; powders of natural organic materials such as starch powders, especially of maize starch, wheat starch or rice starch, which may or may not be crosslinked, such as the starch powders crosslinked with octenyl succinate anhydride, sold under the name Dry-Flo by the company National Starch. Examples of fibres that may be mentioned are polyamide fibres such as especially Nylon 6 fibre (or Polyamide 6) (INCI name: 10 Nylon 6), Nylon 6.6 fibre (or 6.6) (INCI name: Nylon 66) or such as poly-p-Polyamide phenylene terephthamide fibres; and mixtures thereof. These fillers may be present in amounts ranging from 0 to 20% by weight and preferably from 0.5% to 10% by weight relative to the total weight of the composition.

Active agents that may be used especially include vitamins (A, B3, C, E, K, PP, etc.), alone or as a mixture, and also derivatives thereof; keratolytic and/or desquamating agents such as salicylic acid and its derivatives, α-hydroxy acids, for instance lactic acid, citric acid, glycolic acid, and ascorbic acid and its derivatives; antiinflammatory agents; calmatives such as allantoin; depigmenting agents; tensioning agents such as synthetic polymers, plant proteins, polysaccharides of plant origin optionally in the form of microgels, starches, wax dispersions, mixed silicates and colloidal particles of mineral fillers; mattifying agents; agents for preventing hair loss and/or for promoting regrowth of the hair, or anti-wrinkle agents, and mixtures thereof.

The sunscreens may be chosen from organic UV-screening agents, such as the following compounds:
Salicylic derivatives, and especially ethylhexyl salicylate (or octyl salicylate) sold under the trade name Neo Heliopan OS by Haarmann and Reimer;
Dibenzoylmethane derivatives, and especially butyl methoxydibenzoylmethane sold in particular under the trade name Parsol 1789 by Hoffmann LaRoche;
Cinnamic derivatives, and especially ethylhexyl methoxycinnamate sold in particular under the trade name Parsol MCX by Hoffmann LaRoche;
β,β′-Diphenylacrylate derivatives, and especially octocrylene (2-ethylhexyl α-cyano-β,β-diphenylacrylate), sold in particular under the trade name Uvinul N539 by BASF;
Phenylbenzimidazolesulphonic acid;
Benzylidenecamphor derivatives, and especially terephthalylidenedicamphorsulphonic acid manufactured ured under the name Mexoryl SX by Chimex, and 4-methylbenzylidenecamphor sold under the trade name Eusolex 6300 by Merck;
Benzophenone derivatives, and especially benzophenone-3 or Oxybenzone sold under the trade name Uvinul M40 by BASF; benzophenone-4 sold under the trade name Uvinul MS40 by BASF; benzophenone-5;

Phenylbenzimidazole derivatives, and especially Benzimidazilate sold under the trade name Neo Heliopan AP by Haarmann and Reimer;
Triazine derivatives, and especially anisotriazine sold under the trade name Tinosorb S by Ciba Geigy; ethylhexyltriazone sold in particular under the trade name Uvinul T150 by BASF; and diethylhexylbutamidotriazone sold under the trade name Uvasorb HEB by Sigma 3V;
Methylenebis(benzotriazolyl) tetramethylbutyl phenol;
Phenylbenzotriazole derivatives, and especially drometrizole trisiloxane sold under the trade name Silatrizole by Rhodia Chimie; or methylenebis-(benzotriazolyl) tetramethylbutylphenol, sold in solid form under the trade name MIXXIM BB/100 by Fairmount Chemical, or in micronized form as an aqueous dispersion under the trade name Tinosorb M by Ciba Specialty Chemicals;
and mixtures thereof.

The sunscreens may also be chosen from physical sunblocks. As examples of physical sunblocks that may be added to the composition of the invention, mention may be made of pigments and nanopigments of coated or uncoated metal oxides, especially titanium oxide, iron oxide, zirconium oxide, zinc oxide or cerium oxide, and mixtures thereof, these oxides possibly being in the form of optionally coated micro-particles or nanoparticles (nanopigments).

The amount of active agents depends on the desired aim. The active agent(s) may be present, for example, in a concentration ranging from 0.001% to 20%, preferably from 0.01% to 10% and better still from 0.05% to 5% by weight relative to the total weight of the composition.

Needless to say, a person skilled in the art will take care to select the optional compound(s) to be added to the composition according to the invention and the amounts thereof, such that the advantageous properties intrinsically associated with the composition in accordance with the invention are not, or are not substantially, adversely affected by the envisaged addition.

The composition according to the invention may especially be in the form of a more or less fluid cream or a milk, and it may especially constitute a cosmetic or dermatological composition. It then finds its application in a large number of treatments, especially cosmetic treatments of the skin, including the scalp, the hair, the nails, and/or mucous membranes, in particular for caring for, protecting and/or making up body or facial skin, the eyelashes and/or the lips, and/or for haircare (for example a hair mask).

Thus, a subject of the present invention is the cosmetic use of a cosmetic composition as defined above to care for, protect and/or make up the skin and/or the lips and/or for haircare.

A subject of the present invention is also a cosmetic process for treating the skin, including the scalp, the hair and/or the lips, wherein a cosmetic composition as defined above is applied to the skin, the hair and/or the lips.

Moreover, the composition according to the invention is well tolerated by sensitive skin. Thus, a subject of the present invention is also the cosmetic use of a cosmetic composition as defined above, to care for, protect and/or make up sensitive skin.

The compositions according to the invention can be prepared according to conventional techniques: the polymer is dissolved in the aqueous phase. The oily phase is heated to a temperature at which all the solid oily constituents become liquid. At this temperature, the oily phase is introduced with stirring into the neutralized or non-neutralized aqueous phase. The emulsion may be prepared with standard homogenization means, for instance a rotor-stator turbomixer.

The examples below of compositions according to the invention are given by way of illustration and with no limiting nature. The amounts therein are given as percentages by weight, unless otherwise mentioned.

### EXAMPLE 1 ACCORDING TO THE INVENTION

Emulsion comprising a wax and an amphiphilic AMPS copolymer bearing 8.5 mol % of C₁₂₋₁₄(OE)₇ side chains

Oily phase

| | |
|---|---|
| Beeswax | 2% |
| Parleam oil | 10.8% |
| Pentacyclomethicone | 16% |
| Octyl palmitate | 3% |
| Liquid traction of shea butter | 2% |
| Tocopheryl acetate | 0.2% |
| Cetyl alcohol | 3% |
| Stearyl alcohol | 3% |

| Oily phase | |
|---|---|
| Beeswax | 2% |
| Parleam oil | 10.8% |
| Pentacyclomethicone | 16% |
| Octyl palmitate | 3% |
| Liquid traction of shea butter | 2% |
| Tocopheryl acetate | 0.2% |
| Cetyl alcohol | 3% |
| Stearyl alcohol | 3% |
| Aqueous phase | |
| Preserving agent | 1% |
| Triethanolamine at 10% in water | 0.05% |
| Copolymer of AMPS and of Genapol | 0.7% |
| LA-070 methacrylate | (8.5 mol %) |
| Water | qs 100% |

Procedure: The amphiphilic AMPS copolymer is dissolved for 2 hours with stirring in the aqueous phase at 25° C.; the solution obtained is macroscopically homogeneous. The emulsion is prepared by slowly introducing the oily phase, preheated to 70° C., into the aqueous phase with stirring using a Moritz homogenizer at a stirring speed of 2000 rpm for 15 minutes.

The composition obtained is in the form of a soft cream with a viscosity of 39 poises (i.e. 3.9 Pa.s) (measured using a Rheomat 180 machine with a No. 4 spindle and at a shear rate of 200 s⁻¹ after 10 minutes at 25° C.), of pH 5.6. The emulsion is fine under a microscope. After 2 months at room temperature and at 45° C., the emulsion remains fine and conserves the same level of viscosity. It shows no signs of macroscopic destabilization.

Cosmetic Tests:

Example 1 concerns a sensory test on 7 women over 35 years old, with dry or combination dry skin type. A beautician applied the cream, per half-face, to the women's faces. The product is considered as having an unquestionable softening and suppling effect. It has a firm texture and melts quickly on spreading. It is easy to apply, the cream slides well and penetrates slowly without any blocking or sticking effect. To the touch, a slightly waxy soft light film remains on the skin, which gives an impression of protection.

### EXAMPLE2 ACCORDING TO THE INVENTION

Emulsion comprising a wax and an amphiphilic AMPS copolymer bearing 8.5 mol % of C₁₂₋₁₄(OE)₇ side chains

| Oily phase | |
|---|---|
| Petroleum jelly (melting point 53-58° C.) | 3% |
| Silicone gum | 7% |
| Parleam oil | 15.8% |
| Pentacyclomethicone | 9% |
| Octyl palmitate | 3% |
| Palm oil (40/10/50 palmitic-oleic-stearic | 2% |
| acid triglycerides) (melting point 37° C.) | |
| (wax) | |
| Cetyl alcohol | 1% |
| Stearyl alcohol | 13% |
| Aqueous phase | |
| Preserving agent | 0.6% |
| Triethanolamine at 10% in water | 0.06% |
| Glycerol | 5% |
| Copolymer of AMPS and of Genapol | 0.8% |
| LA-070 methacrylate (8.5 mol %) | |
| Water | qs 100% |

Aqueous phase

| | |
|---|---|
| Preserving agent | 0.6% |
| Triethanolamine at 10% in water | 0.06% |
| Glycerol | 5% |
| Copolyrner of AMPS and of Genapol | 0.8% |
| LA-070 methacrylate (8.5 mol %) | |
| Water | qs 100% |

Procedure: identical to that of Example 1.

The composition is in the form of a soft cream with a viscosity of 27 poises (i.e. 2.7 Pa.s) (measured using a Rheomat 180 machine with a No. 3 spindle and at a shear rate of 20 s⁻¹ after 10 minutes at 25° C.), of pH 6.5. The emulsion is fine under a microscope. After 2 months at room temperature and at 45° C., the emulsion remains fine and conserves the same level of viscosity. It shows no signs of macroscopic destabilization.

### EXAMPLE 3 ACCORDING TO THE INVENTION

Emulsion comprising a wax and an amphiphilic AMPS copolymer bearing 7.35 mol % of C₁₆₋₁₈(OE)₈ side chains

| Oily phase | |
|---|---|
| Parleam oil | 15.8% |
| Pentacyclomethicone | 16% |
| Octyl palmitate | 3% |
| Liquid fraction of shea butter | 2% |
| Beeswax | 1% |
| Tocopheryl acetate | 0.2% |
| Cetyl alcohol | 1% |
| Stearyl alcohol | 1% |
| Aqueous phase | |
| Preserving agent | 1% |
| Triethanolamine at 10% in water | 0.03% |
| Copolymer of AMPS and of Genapol | 0.5% |
| LA-080 methacrylate (7.35 mol %) | |
| Water | qs 100% |

Aqueous phase

| | |
|---|---|
| Preserving agent | 1% |
| Triethanolamine at 10% in water | 0.03% |
| Copolymer of AMPS and of Genapol | 0.5% |
| LA-080 methacrylate (7.35 mol %) | |
| Water | qs 100% |

Procedure: identical to that of Example 1.

The composition is in the form of a soft cream with a viscosity of 13.4 poises (i.e. 1.34 Pa.s) (measured using a Rheomat 180 machine with a No. 3 spindle and at a shear rate of 200 s⁻¹ after 10 minutes at 25° C.), of pH 5.8. The emulsion is fairly fine under a microscope. After 2 months at room temperature and at 45° C., the emulsion remains fairly fine and conserves the same level of viscosity. It shows no signs of macroscopic destabilization.

### EXAMPLE 4 ACCORDING TO THE INVENTION

Emulsion comprising a wax and an amphiphilic AMPS copolymer bearing 7.35 mol % of C₁₆-₁₈(OE)₈ side chains

| Oily phase | |
|---|---|
| Parleam oil | 15.8% |
| Pentacyclomethicone | 16% |
| Octyl palmitate | 3% |
| Liquid fraction of shea butter | 2% |
| Beeswax | 1% |
| Tocopheryl acetate | 0.2% |
| Cetyl alcohol | 1% |
| Stearyl alcohol | 1% |
| Aqueous phase | |
| Preserving agent | 0.6% |
| Triethanolamine at 10% in water | 0.03% |
| Glycerol | 5% |
| Copolymer of AMPS and of Genapol | 0.5% |
| T-080 methacrylate (7.35 mol %) | |
| Water | qs 100% |
| Nylon powder | 3% |

Aqueous phase

| | |
|---|---|
| Preserving agent | 0.6% |
| Triethanolamine at 10% in water | 0.03% |
| Glycerol | 5% |
| Copolymer of AMPS and of Genapol | 0.5% |
| T-080 methacrylate (7.35 mol %) | |
| Water | qs 100% |
| Nylon powder | 3% |

Procedure: identical to that of Example 1, with addition of nylon powder after preparation of the emulsion.

The composition is in the form of a soft cream with a viscosity of 18.5 poises (i.e. 1.85 Pa.s) (measured using a Rheomat 180 machine with a No. 3 spindle and at a shear rate of 200 s⁻¹ after 10 minutes at 25° C.), of pH 6. The emulsion is fairly fine under a microscope. After 2 months at room temperature and at 45° C., the emulsion remains fairly fine and conserves the same level of viscosity. It shows no signs of macroscopic destabilization.

### EXAMPLE 5 ACCORDING TO THE INVENTION

Emulsion comprising a wax and an amphiphilic AMPS copolymer bearing 8.5 mol % of C₁₂₋₁₄(OE)₇ side chains

| Oily phase | |
|---|---|
| Beeswax | 2% |
| Parleam oil | 13% |
| Aqueous phase | |
| Preserving agent | 1% |
| Triethanolamine at 10% in water | 0.06% |
| Copolymer of AMPS and of Genapol | 1% |
| LA-070 methacrylate (8.5 mol %) | |
| Water | qs 100% |

Aqueous phase

| | |
|---|---|
| Preserving agent | 1% |
| Triethanolamine at 10% in water | 0.06% |
| Copolymer of AMPS and of Genapol | 1% |
| LA-070 methacrylate (8.5 mol %) | |
| Water | qs 100% |

Procedure: identical to that of Example 1.

The composition obtained is in the form of a milk (pH approximately 6.1). The mean droplet size is 10 μm. Its viscosity, measured using a Rheomat 180 machine at 25° C. at a shear rate of 200 s⁻¹ using a No. 2 spindle, is 0.13 Pa.s. After 24 hours at 25° C., this emulsion remains stable.

### COMPARATIVE EXAMPLE 1

Wax-free emulsion containing an amphiphilic AMPS copolymer bearing 8.5 mol % of C₁₂₋₁₄(OE)₇ side chains

| Oily phase | |
|---|---|
| Cyclopentadimethylsiloxane | 6% |
| Parleam oil | 9% |
| Aqueous phase | |
| Preserving agent | 1% |
| Triethanolamine at 10% in water | 0.06% |
| Copolymer of AMPS and of Genapol | 1% |
| LA-070 methacrylate (8.5 mol %) | |
| Water | qs 100% |

Procedure: identical to that of Example 1.

The formula is in the form of a milk that becomes instantaneously destabilized, with the appearance of a creaming phenomenon (separation of some of the oily phase). This emulsion comprising an amphiphilic AMPS copolymer bearing 8.5 mol % of C₁₂₋₁₄(OE)₇ side chains is unstable in the absence of wax, whereas Examples 1, 2 and 5 according to the invention, which comprise the same polymer and a wax, are stable.

### COMPARATIVE EXAMPLE 2

Emulsion with wax containing an amphiphilic AMPS copolymer bearing 3.55 mol % of C₁₆₋₁₈(OE)₂₅ side chains

| Oily phase | |
|---|---|
| Parleam oil | 15.8% |
| Pentacyclomethicone | 16% |
| Octyl palmitate | 3% |
| Liquid fraction of shea butter | 2% |
| Beeswax | 1% |
| Tocopheryl acetate | 0.2% |
| Cetyl alcohol | 1% |
| Stearyl alcohol | 1% |
| Aqueous phase | |
| Preserving agent | 1% |
| Triethanolamine at 10% in water | 0.03% |
| Copolymer of AMPS and of Genapol | 0.5% |
| T-250 methacrylate (3.55 mol %) | |
| Water | qs 100% |

The emulsion obtained is heterogeneous and has an oily phase that separates out from the aqueous phase and floats above the emulsion, whereas Example 3, which is identical to this example in all respects with the exception of the polymer used, is entirely stable. This example shows that polymers comprising 25 oxyethylene groups do not allow a stable O/W emulsion to be obtained.

### COMPARATIVE EXAMPLE 3

Emulsion with wax containing an amphiphilic AMPS copolymer bearing 58.2 mol % of C₁₂₋₁₄(OE)₇ side chains

| Oily phase | |
|---|---|
| Parleam oil | 15.8% |
| Pentacyclomethicone | 16% |
| Octyl palmitate | 3% |
| Liquid fraction of shea butter | 2% |
| Beeswax | 1% |
| Tocopheryl acetate | 0.2% |
| Cetyl alcohol | 1% |
| Stearyl alcohol | 1% |
| Aqueous phase | |
| Preserving agent | 1% |
| Triethanolamine at 10% in water | 0.03% |
| Copolymer of AMPS and of Genapol | 0.5% |
| LA-070 methacrylate (58.2 mol %) | |
| Water | qs 100% |

The emulsion obtained is heterogeneous and shows a creaming phenomenon after 24 hours at room temperature (separation of the aqueous and oily phases), whereas Examples 1, 2 and 5, which comprise an identical polymer but with 8.5% of hydrophobic chains, are entirely stable. This example shows that polymers comprising more than 25% of hydrophobic chains do not allow a stable O/W emulsion to be obtained.

1. A composition suitable for topical application comprising an oily phase dispersed in an aqueous phase, at least one wax, and at least one non-crosslinked amphiphilic polymer, said polymer comprising from:
(a) 80 mol % to 99 mol % of 2-acrylamido-2-methylpropane-sulphonic acid (AMPS) units of formula (I) below: 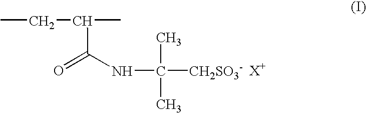
in which X⁺ is a proton, an alkali metal cation, an alkaline-earth metal cation, an ammonium ion or an organic cation; and
(b) 1 mol % to 20 mol % of units of formula (ii) below: 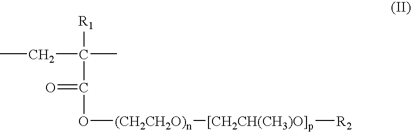
in which n and p, independently of each other, denote an integer ranging from 0 to 24, with the proviso that n+p is less than 25; R₁, denotes a hydrogen atom or a linear or branched alkyl radical containing from 1 to 6 carbon atoms, and R₂ denotes a linear or branched alkyl radical containing from 6 to 30 carbon atoms.

2. The composition according to claim 1, wherein the polymer is partially or totally neutralized with a mineral or organic base.

3. The composition according to claim 1, wherein the polymer comprises:
(a) 85 mol % to 99 mol % of 2-acrylamido-2-methylpropane-sulphonic acid (AMPS) units of formula (I), and
(b) 1 mol % to 15 mol % of units of formula (III): 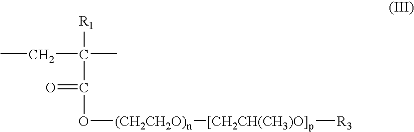
in which n and p, independently of each other, denote an integer ranging from 7 to 24, with the proviso that n+p is less than 25; R₁ denotes a hydrogen atom or a linear or branched alkyl radical containing from 1 to 6 carbon atoms, and R₃ denotes a linear or branched alkyl radical containing from 6 to 15 carbon atoms.

4. The composition according to claim 1, wherein the polymer comprises units of 2-acrylamido-2-methylpropanesulphonic acid (AMPS) or a sodium or ammonium salt thereof, and units obtained by reaction of a (meth) acrylic acid ester and one of the following:
a C₁₀-C₁₈ alcohol oxyethylenated with 8 mol of ethylene oxide,
a C₁₁ oxo alcohol oxyethylenated with 8 mol of ethylene oxide,
a C₁₁oxo alcohol oxyethylenated with 7 mol of ethylene oxide,
a C₁₂-C₁₄ alcohol oxyethylenated with 7 mol of ethylene oxide,
a C₁₂-C₁₄ alcohol oxyethylenated with 9 mol of ethylene oxide,
a C₁₂-C₁₄ alcohol oxyethylenated with 11 mol of ethylene oxide,
a C₁₆-C,₁₈ alcohol oxyethylenated with 8 mol of ethylene oxide,
a C₁₆-C,₁₈ alcohol oxyethylenated with 15 mol of ethylene oxide,
a C₁₆-C₁₈ alcohol oxyethylenated with 11 mol of ethylene oxide,
a C₁₆-C₁₈ alcohol oxyethylenated with 20 mol of ethylene oxide.

5. The composition according to claim 4, wherein the polymer is a copolymer comprising the following units:
91.5 mol % of AMPS and 8.5 mol % of a C₁₂-C₁₄ alkyl methacrylate comprising 7 oxyethylene groups; or 92.65 mol % of AMPS and 7.35 mol % of a C₁₆-C₁₈ alkyl methacrylate comprising 8 oxyethylene groups.

6. The composition according to claim 1, wherein the amount of amphiphilic polymer(s) ranges from 0.05% to 20% by weight relative to the total weight of the composition.

7. The composition according to claim 1, wherein the oily phase represents from 15% to 75% by weight relative to the total weight of the composition.

8. The composition according to claim 1, wherein the amount of wax(es) ranges from 0.1% to 10% by weight relative to the total weight of the composition.

9. The composition according to claim 1, wherein the wax is selected from the group consisting of mineral waxes, waxes of animal origin, waxes of plant origin, hydrogenated oils that are solid at 25° C., fatty esters and glycerides that are solid at 25° C., synthetic waxes and silicone waxes, and mixtures thereof.

10. The composition according to claim 1, wherein the wax is selected from the group consisting of carnauba wax, polyethylene waxes with a starting melting point of greater than 65° C., microcrystalline waxes with a starting melting point of greater than 65° C., and mixtures thereof.

11. The composition according to claim 1, wherein said composition is substantially free of emulsifying surfactant.

12. The composition according to claim 1, wherein said composition constitutes a cosmetic or dermatological composition.

13. A method to care for, protect and/or make up the skin and/or the lips and/or for haircare, comprising application of the composition of claim 1 to the skin, lips or hair.

14. A method to care for, protect and/or make up sensitive skin, comprising application of the composition of claim 1 to sensitive skin.

15. The composition according to claim 1, wherein said composition, after storage for 24 hours at all temperatures between 4° C. and 50° C., shows no macroscopic change in colour, odour or viscosity and no variation in pH.

16. The composition according to claim 1, wherein said composition has a viscosity at 25° C. of 0.1 to 25 Pa.s as measured with a Rheomat 180 machine.

17. The composition according to claim 1, wherein said polymer has a weight-average molecular weight of 10,000 to 10,000,000.
